(19) United States
(12) Patent Application Publication
(10) Pub. No.: US 2008/0269223 A1
(43) Pub. Date: Oct. 30, 2008

(54) Mouth Dissolvable and Meltable, and Water Dispersable Delivery Formulation

(76) Inventors: Saibal Chakravorty, Mumbai (IN); V. Hariharan, Tamil Nadu (IN)

Correspondence Address: LADAS & PARRY LLP, 224 SOUTH MICHIGAN AVENUE, SUITE 1600, CHICAGO, IL, 60604, US

(21) Appl. No.: 11/547,297

(22) PCT Filed: Apr. 4, 2005
(86) PCT No.: PCT/IN05/00101

§ 371 Date: Oct. 4, 2006

(30) Foreign Application Priority Data
Apr. 6, 2004 (IN) 419/MUM/2004

(51) Int. Cl. A61K 31/53

(52) U.S. Cl. 514/242

(57) ABSTRACT

A mouth dissolvable and meltable, and water dispersable delivery system for oral administration consisting of an antiepileptic drug, one or more swelling agents, one or more of fillers, one or more of disintegrating agents, and one or more of binders is disclosed. The swelling agent is powdered cellulose, filler is spray dried mannitol, disintegrating agent is crosslinked polyvinyl pyrrolidone and binder is maltodextrin. This system optionally comprises one or more of other excipients selected from the group comprising lubricants, sweetners and flavouring agent.

### FIELD OF THE INVENTION

The present invention relates to a mouth dissolvable and meltable, and water dispersable delivery system. Particularly, it relates to a mouth dissolvable and meltable, and water dispersable delivery system anti-epileptic drugs. More particularly, it relates to a mouth dissolvable and meltable, and water dispersable delivery system for Lamotrigine.

### BACKGROUND OF INVENTION

Epileptic seizures are common and are treated in all branches of Medicine. Approximately 10% of the population will have one or more seizures during their lifetime. Seizures are symptoms that occur in acute illness i.e. provoked seizures or in epilepsy i.e. unprovoked seizures. Epilepsy is characterized by unprovoked, recurring seizures that disrupt the nervous system and can cause mental and physical dysfunction. About 14% of epilepsy patients are under 15 years old and 24% are over 64, with 62% being between those ages.

Epilepsy is not a single disorder but rather a wide spectrum of problems. It is an uncontrolled electrical discharge from nerve cells in the cerebral cortex, which is the part of the brain that integrates higher mental functions, general movement, and the functions of the internal organs in the abdominal cavity, perception, and behavioral reactions. Epilepsy types are generally grouped as partial or generalized seizures. Partial, or focal, seizure is the more common type of epilepsy and is generally defined as a disorder of the neurons that starts on one side of the brain. It is currently subcategorized as “simple” and “complex partial” seizures.

Generalized seizures are caused by nerve cell disturbances that occur in more diffuse areas of the brain than do partial seizures. Therefore, they have a more serious effect on the patient. They are further subcategorized as tonic-clonic (or grand mal) or absence (petit mal) seizures. Tonic-Clonic (Grand Mal) Seizures. The first stage of a grand mal seizure is called the tonic phase, in which the muscles suddenly contract, causing the patient to fall and lie rigidly for about 10 to 30 seconds. Some people experience a premonition or aura before a grand mal seizure; most, however, lost consciousness without warning.

Spasms occur for about 30 seconds to a minute as the seizure enters the clonic phase, when the muscles begin to alternate between relaxation and rigidity. After this phase, the patient may lose bowel or urinary control. The seizure usually lasts a total of two or three minutes, after which the patient remains unconscious for a while and then awakens to confusion and extreme fatigue.

Absence (Petit Mal) Seizures. Petit mal or absence seizures are brief (3 to 30 seconds) and may consist of only a short cessation of physical movement and loss of attention. About 25% of patients with petit mal develop grand mal seizures.

Status Epilepticus. Status epilepticus is often defined as recurrent convulsions that last for more than 20 minutes and are interrupted by only brief periods of partial relief. Some experts believe these criteria are too strict and that the condition should be diagnosed if seizures last at least five minutes or more, when the patient does not fully recover consciousness between two or more seizures. Although any type of seizure can be sustained or recurrent, the most serious form of status epilepticus is the generalized convulsive or tonic-clonic type. In more than a third of cases, status epilepticus occurs with the first seizure.

The trigger is often unknown, but can include the following:—
Failure to take anti-epileptic medications (comprising about a third of these events).
Abrupt withdrawal of certain anti-epileptic drugs, particularly barbiturates and benzodiazepines.
High fever
Poisoning
Electrolyte imbalances (imbalance in calcium, sodium and potassium)
Cardiac arrest
Stroke. In one study, about 9% of stroke patients with seizures had status epilepticus, which was associated with higher disability after the stroke, particularly if these severe seizures occurred within a week of the stroke.
Low blood sugar in people with diabetes.

Status epilepticus (SE) is a serious condition that can lead to chronic epilepsy and can be life threatening. Permanent brain damage or death can result if the seizure is not treated effectively; the longer the seizure lasts, the greater the danger. Mortality rates from this condition are about 10%. (This high mortality rate is most likely due to a high incidence of myoclonic SEs in elderly adults after cardiac arrest. One study reported much lower mortality rates from SE when cardiac arrest in elderly epilepsy patients is excluded.)

The treatment goals of status epilepticus are to stop the seizures, prevent recurrence, determine and prevent any factors that might have triggered it and manage any complications.

Initial Management. The earlier a patient is treated, the better the results. In one study, seizures stopped in 80% of patients who were treated within 30 minutes. Only 40% of patients responded when they were treated after two hours.

Lamotrigine is effective as add-on therapy and is well tolerated in treating partial and generalized seizures. It has now been approved as monotherapy for partial seizures in adults who have not responded to standard agents. (It has not yet been approved as first-line therapy.) Recent studies have suggested that it is as effective as carbamazepine and phenytoin and patients' tolerate it better. It is currently approved in children only for Lennox astaut syndrome but is proving to be effective and tolerable for children with a variety of other seizure types. Lamotrigine is a drug of phenyltriazine class chemically unrelated to the existing antiepileptic drugs (AED). Lamotrigine may be chemically named 3,5-diamino-b-[2,3-dichlorophenyl]-1,2,4-triazine and is disclosed in U.S. Pat. No. 4,602,017 and European Patent 0021121. Lamotrigine's primary action is to modulate voltage-gated sodium channels. Evidence suggests that it decreases glutamate transmission, directly reduce calcium influx, mildly blocks transmitter re uptake and alters intracellular mechanisms of resting transmitter release.

Lamotrigine is capable of absorbing completely following oral administration, reaching peak plasma concentration in 1.4 to 4.8 hours (Tₘₐₓ). The average half life of Lamotrigine is approximately 24 hours but decreases to approximately 7.2 hours, when used concurrently with phenytoin and increases to approximately 59 hours with Valproic Acid. The absolute bioavailability of oral Lamotrigine is found to be 98%. The bioavailability of Lamotrigine is not affected by food Lamotrigine is approximately 55% bound to human plasma protein. It does not displace other anti-epileptic drugs such as carbamazepine, phenytoin, phenobarbitol from protein binding sites. Lamotrigine is primarily metabolised to the inactive N-2 nitrogen glucoronide metabolite by the enzyme uridine diphosphate glucorosyl transferase (UGT). UGT increases the polarity of lipophilic substrates, often leading to their loss of biological activity and enhanced elimination from the body. The adverse effects of Lamotrigine are infrequent when the drug is used alone but become frequent when the drug is combined with other anti-convulsants. While most of the rashes are mild, approx. 1 in 500 patients develop exfoliative dermatitis. A slow upward dose titration is suggested or recommended to reduce the incidence of serious rash, which may delay the attainment of adequate dosage for 6 weeks. Lamotrigine has positive effects on cognitive function, but occasionally produce insomnia. Hence, based on these facts Lamotrigine was selected as preferable anti-epileptic drug for the presently disclosed delivery formulation.

The drug delivery systems existing in the market are either in tablet, capsule, liquid, suppositories, nasal spray, transdermal patch form or parenteral form. But for the anti-epileptic drug, particularly for Lamotrigine, the requirement is to have a system, which cannot only get dispersed in water, but also get dissolve and melt in mouth on contact with saliva. Such dissolvation or melting in oral cavity is expected to be as early as possible, that is, in minimum possible duration, preferably in less than 3 minutes.

The advantage of mouth dissolvable and meltable drug delivery systems is to have accurate dose delivery and ease to administer with minimum biovariant. Further, advantage of such drug delivery system is that it avoids chances of any type of contamination due to being no-touch facility. The doctor can directly place the drug delivery system, which may be in any form—tablet or liquid form in the oral cavity of the patient without any touch or contact with the drug component. Similarly, the patient can himself place the drug delivery system, which may be in any form—tablet or liquid form in the oral cavity without any touch or contact with the drug component. Further, this system can also overcome common disadvantages of the conventional drug delivery systems including compliance issues, adverse drug reactions, inadequate bioavailability due to the way the drug is delivered.

Similarly, the water dispersion of anti-epileptic drug, particularly Lamotrigine should also be as early as possible, that is in minimum possible duration, preferably in less than 1 minute. But the disadvantage of such drug delivery system is that it leave residue of the dispersed medium in the container of dispersion and hence does not result in accurate drug delivery. Further, there is chance of contamination due to use of a dispersion medium. Further, the inconvenience of swallowing oral tablet dosage form is prevalently encountered by both elderly and young individuals, which can be avoided by dispersing the tablet in water and administering to the patient instantaneously.

This type of suspension form of administration can be undesirable in patients, who are on reduced liquid intake plans, who have limited access to water, who are travelling and also cause variation in the amount of drug delivered from the dosage form to the patient. Hence, causing bioavailability problems.

Accordingly, the most preferred drug delivery system is mouth dissolvable and meltable drug delivery system, but the scope of water dispersion drug delivery system is not eliminated. However, the drug delivery systems for anti-epileptic drug, particularly for Lamotrigine available in the market are in conventional tablet form or in water dispersible tablet form. Some of the water dispersible drug delivery systems state that these can be used as mouth dissolvable systems, but these systems have not been established as mouth dissolvable and meltable systems.

Accordingly, this is the main object of the present invention to have a drug delivery system particularly for anti-epileptic drug, more particularly for Lamotrigine drug, which can not only get dispersed in water in minimum possible duration, preferably in less than 1 minute, but can also get dissolved and melt in mouth on contact with saliva in minimum possible duration, preferably in less than 3 minutes, that is can also deliver the desired drug, particularly the Lamotrigine at pH 7.2, the approximate pH of saliva and overcome disadvantages of the conventional drug delivery systems and water dispersible systems.

The known water dispersible tablets of Lamotrigine describe the designing of the process of Lamotrigine being granulated in a vehicle of swellable clay with microcrystalline cellulose as a filler and polyvinyl pyrrolidone as binder in alcoholic medium. The main ingredient of this formulation is pharmaceutically acceptable swellable clay, particularly aluminium magnesium silicate and most preferably Veegum. The other swellable clay used is bentonite (GB2278057). These ingredients are geographically oriented, that is these are found in regions of Canada and US. Therefore, these ingredients have restriction in availability. Further, these are processed to remove grit and non-swellable materials before using in the pharmaceutical materials. Accordingly, there is need to develop a drug delivery system without the swellable clay, particularly without Veegum and/or bentonite.

Further, the main requirement of the disclosed dosage form of GB2278057 is that the swellable clay is present within the granules, that is intra-granular, of the tablet comprising up to 90% of the Lamotrigine and up to 60%, most preferably about 5% of the swellable clay. This delivery system is based on the findings that very low intra-granular amounts, such as 1% w/w or below, and higher extra-granular amounts, such as about 10% w/w or higher may decrease the dispersion time, but in general extra-granular addition has little or no effect on the dispersion time. This delivery system with maximum percentage of swellable clay present within the granules, that is present intra-granularly in the drug and optionally the granules outside the drug, that is extra-granularly in the drug has been found to be limited by other practical consideration such as poor flow and compression properties.

However, the present inventors have surprisingly found that the presence of vehicle granules, which in the present invention has been used as a swelling agent extra-granularly to the granules of the drug, particularly Lamotrigine, that is when the drug particles are present within the particles of the selected swelling agent have shown desirable and promising results. The dispersion time has been found to be less than 1 minute. Further, this drug delivery system has shown desired dispersion characteristics at pH of about 7.2, which is pH of saliva, which is indicative of the fact that this drug delivery system is expected to have better mouth dissolvability. The melting point depression studies have also revealed that it shows melting point depression from about 215° C. to about 190° C. Accordingly, the presently disclosed delivery system also shows better meltability characteristics. The swellability studies of the present delivery system have further revealed that it shows swellability (volume expansion) of the drug, particularly of Lamotrigine in the selected vehicle system of selected swelling agent about four times the swellability observed in other vehicle systems which is an indication of better mouth dissolvability.

The other drug delivery system for anti-epileptic drugs, particularly for Lamotrigine has been disclosed in U.S. Pat. No. 5,555,639. This drug delivery system is also based on above-findings and vehicle of swellable clay. Further, the above drug delivery systems are manufactured by adding the swellable clay only during the granulation to have the granules of the swellable clay within the granules of the drug and to have water dispersible drug delivery system.

However, the present invention is based on the adsorbent property of the selected vehicle (swelling agent) which in combination with selected filler first adsorbs the drug, particularly the Lamotrigine in the fibrous orifice. It has been surprisingly found that this system easily releases the drug after drying and on contact with aqueous medium, which is saliva when the present drug delivery system is administered directly in the mouth and water when the present drug delivery system is administered after dissolution in water. Accordingly, the present drug delivery system has been found to exhibit both the characteristics—mouth dissolvability and meltability as well as dispersibility in water.

### NEED OF THE INVENTION

Accordingly, there is a need to develop a drug delivery system for anti-epileptic drug, particularly for Lamotrigine drug, which cannot only get dispersed in water in minimum possible duration, preferably in less than 1 minute, but can also get dissolved and melt in mouth on contact with saliva in minimum possible duration, preferably in less than 3 minutes, that is can also deliver the desired drug, particularly the Lamotrigine at pH 7.2, the approximate pH of saliva and overcome disadvantages of the conventional drug delivery systems and water dispersible systems.

In a view to achieve the above desired characteristics of the drug delivery system for anti-epileptic drug particularly for Lamotrigine, an extensive research work has been carried out with different excipients and process designs to develop an efficient drug delivery system, which would overcome the above-mentioned disadvantages of the prior art.

The drug delivery system to be designed and developed should thereby—
require no water for oral administration, yet dissolves or melts rapidly in the mouth on contact with saliva;
allow high drug accuracy and drug loading;
compatible with taste masking and provide a pleasant mouth feel;
leave no residue in the mouth after oral administration;
exhibit low sensitivity to environmental conditions such as humidity and temperature; and
also provide a dispersion in accordance with the test for dispersible tablets defined in the British Pharmacopoeia.

Accordingly, the drug delivery system to be designed and developed should have dual capability of administration, that is not only to be administered after dispersing in water, but also to be simply taken directly as mouth dissolving and meltable tablet, thereby improving accurate drug delivery and patient compliance. The pharmaceutical acceptability of the system developed would depend on the efficacy of the dosage form to result into compliance of all the quality aspects comprising controlling devices to predict a definite behaviour pattern of the system.

### OBJECTIVE OF THE INVENTION

As described hereinabove, the present invention is aimed to have an improved drug delivery system for a drug, particularly for an anti-epileptic drug, more particularly for Lamotrigine which should overcome at least some of the above-described disadvantages, limitations and drawbacks of relevant prior art and provide above-described of the present invention and above-defined desired characteristics.

Main object of the present invention to have a drug delivery system particularly for anti-epileptic drug, more particularly for Lamotrigine drug, which can not only get dispersed in water in minimum possible duration, preferably in less than 1 minute, but can also get dissolved and melt in mouth on contact with saliva in minimum possible duration, preferably in less than 3 minutes, that is can also deliver the desired drug, particularly the Lamotrigine at pH 7.2, the approximate pH of saliva and overcome disadvantages of the conventional drug delivery systems and water dispersible systems.

Another object is to have a drug delivery system which shows better melting point depression and swellability properties of the active ingredient.

Still another object of the present invention is to have a drug delivery system which can easily release the drug on contact with aqueous medium, which can be either saliva or water

Yet another object of the present invention is to have a drug delivery system, which allows high drug accuracy and drug loading, and is compatible with taste masking and provide a pleasant mouth feel.

This is still an object of the present invention to have a drug delivery system which leave no residue in the mouth after oral administration and exhibits low sensitivity to environmental conditions such as humidity and temperature.

This is still another object of the present invention to have a drug delivery system which has improved patient compliance.

This is yet an object of the present invention to have a drug delivery system which facilitate accurate dispersion of the drug with enhanced bioavailability and bioabsorption, and makes the absorption area available for enhanced bioavailability in the upper part of the G. I. Tract, and making the drug less susceptible to first pass metabolism in the liver.

This is yet another object of the present invention to have a drug delivery system which can be manufactured in a non-alcoholic system.

### BRIEF DESCRIPTION OF INVENTION

Accordingly, the present invention relates to a mouth dissolvable and meltable, and water dispersable delivery formulation for oral administration consisting of an antiepileptic drug, one or more swelling agents, one or more of fillers, one or more of disintegrating agents, and one or more of binders, characterized in that said antiepileptic drug is preferably Lamotrigine for the antiepileptic treatment; said swelling agent is selected from the group comprising powdered cellulose and crosscarmellose sodium; said filler is selected from the group comprising spray dried mannitol, glucose, sorbitol, maltose and fructose; said disintegrating agent is selected from the group comprising crosslinked polyvinyl pyrrolidone, corn starch, acacia, crosscarmellose sodium and xanthan gum; and said binder is maltodextrin; and the formulation may optionally comprise one or more of other excipients selected from the group comprising lubricants, sweetners and flavouring agent.

The present invention provides a mouth dissolving delivery system, such as a tablet, that can be employed in the pharmaceutical field using an antiepileptic drug, namely Lamotrigine which is used in the treatment of status epilepticus and also as an adjunctive therapy in simple partial seizures and secondarily generalised tonic-clonic seizures.

It has been observed that the present system comprising an anti-epileptic drug in a vehicle of powdered cellulose and crosscarmellose sodium as one or more swelling agent; and spray dried mannitol, glucose, sorbitol, maltose and fructose as one or more fillers; crosslinked polyvinyl pyrrolidone, corn starch, acacia, crosscarmellose sodium and xanthan gum as one or more disintegrating agent; and maltodextrin as binder result in release of the drug immediately on contact with aqueous medium of pH about 1.2 to about 7.2.

The invention involved in the development of an improved oral solid dosage form which upon ingestion would immediately “dissolve” or “melt” when placed in mouth on coming in contact with saliva. This invention would help better compliance in the treatment of epilepsy as the dosage form can be taken either without water or with water.

The other features of the presently disclosed drug delivery system includes without limiting the scope of the present invention—increased bioavailability in minimal time due to increased absorption area with reduction of particle size of the drug, which in accordance with the present invention has been preferably selected at about ≦100 microns, more preferably at about ≦70 microns and most preferably at about ≦40 microns, and it provides accurate dose as it is uniform in both weight and active ingredient content and can be consumed without any measurement at the time of it's use, i.e., also without causing any spoilage when taken out of its package.

The other features and the preferred embodiments of the present invention have been described with the help of accompanying figures, which are not intended to restrict scope of the present invention.

### DESCRIPTION OF THE FIGURES

FIG. 1 shows the compositions of formulations prepared in accordance to the preferred and best embodiments of the present invention.

FIG. 2 shows a comparative In-vitro dissolution profile of the drug delivery system in accordance to one of the preferred embodiments of the present invention in 100 mg tablet form employing unmicronised and micronised Lamotrigine having particle size of about ≦40 microns.

FIG. 3 shows a comparative In-vitro dissolution profile of the drug delivery system in accordance to one of the preferred embodiments of the present invention in 25 mg, 100 mg and 200 mg tablet form.

FIG. 4 shows a comparison of In-vitro dissolution profile of the drug delivery system in accordance to one of the preferred embodiments of the present invention in 200 mg tablet form in discriminatory dissolution medium of pH 4.5 and pH 7.2.

FIG. 5 shows results of melting point depression studies of Lamotrigine in combination with α-form of spray dried mannitol which are taken in a ratio of 1:1 in accordance to one of the preferred embodiments of the present invention. The two graphs in this figure have been plotted as against the melting point of α-form of spray dried mannitol.

FIG. 6 shows results of swellability studies in accordance to one of the preferred embodiments of the present invention which have been measured by way of volume expansion of Lamotrigine with powdered cellulose, with hydroxypropyl cellulose (L) and with sodium carboxymethyl cellulose which are taken in a ratio of 1:1.

FIG. 7 shows comparative In-vitro dissolution profile of Lamotrigine in 200 mg tablet form which in accordance to one of the preferred embodiments of the present invention is taken in a vehicle of powdered cellulose of average particle size about 60 microns v/s. 200 microns.

FIG. 8 shows comparative In-vitro dissolution of Lamictal 200 mg of M/s. Glaxo Wellcome, UK with present drug delivery system comprising Lamotrigine in 200 mg tablet form in accordance to one of the preferred embodiments of the present invention in discriminatory dissolution media of pH 4.5.

FIG. 9 shows comparative In-vitro dissolution of Lamictal 2 mg of M/s. Glaxo Wellcome, UK with present drug delivery system comprising Lamotrigine in 2 mg tablet form in accordance to one of the preferred embodiments of the present invention in discriminatory dissolution media of pH4.5.

FIG. 10 shows IR spectra of a-form of mannitol.

The other preferred embodiments of present invention will become more apparent when the following description is read with the accompanying figures and following examples.

### DETAILED DESCRIPTION OF THE INVENTION

The present invention relates to a mouth dissolvable and meltable, and water dispersable delivery formulation for oral administration consisting of an antiepileptic drug, one or more swelling agents, one or more of fillers, one or more of disintegrating agents, and one or more of binders, characterized in that the antiepileptic drug is preferably Lamotrigine for the antiepileptic treatment; the swelling agent is selected from the group comprising powdered cellulose and crosscarmellose sodium; the filler is selected from the group comprising spray dried mannitol, glucose, sorbitol, maltose and fructose; the disintegrating agent is selected from the group comprising crosslinked polyvinyl pyrrolidone, corn starch, acacia, crosscarmellose sodium and xanthan gum; and the binder is maltodextrin; and wherein said formulation may optionally comprises one or more of other excipients selected from the group comprising lubricants, sweetners and flavouring agent.

In accordance with present invention, the Lamotrigine is micronised Lamotrigine which has shown better dissolution profile. Further, the Lamotrigine has particle size of about ≦100 micron, preferably about ≦70 micron, most preferably about ≦40 micron.

The swelling agent in accordance with the invention is powdered cellulose [Arbocel®] which is preferably fibrous and derived from plant, and has average particle size of about 200 microns, most preferably of about 60 microns. It has been surprisingly observed that the powdered cellulose having average particle size of about 60 microns has shown better In-vitro dissolution profile than the powdered cellulose having higher average particle size of about 200 micrins. The powdered cellulose in accordance to the present invention has bulk density of about 0.1 to 0.28 gm/ml, pH of about 5 to 7.5, ether and water soluble substance of about ≦0.15% and about ≦1.5% respectively, with loss on drying about ≦6%, and degree of polymerisation of about 440 to 2250.

The filler is mannitol, preferably a form of spray dried mannitol. The present inventors have surprisingly found that the α form of spray dried mannitol shows better dissolution properties as compared to other forms of mannitol. Accordingly, α form of spray dried mannitol is most preferred in accordance to the present invention. The α-form of mannitol has mean particle size diameter of about 150 micron and Haussner number of about 1.16, having dissolution time in water with a ratio of 1:30 with respect to water in w/w, not more than 5 seconds at 20° C.

In accordance to the present invention, the disintegrating agent consists of crosslinked polyvinyl pyrrolidone or crosscarmellose sodium or both; the maltodextrin [Glucidex®] has dextrose equivalent of about 13 and protein content of about ≦0.15%, carbohydrate composition of glucose of about 1%, maltose of about 2%, and oligo and polysaccharides of about 97%.

The presently disclosed drug delivery system can be further combined with one or more excipients selected from the group comprising lubricants, sweetners and flavouring agents. The lubricating agent is selected from a group comprising calcium stearate, colloidal silicon dioxide and talc. The sweetners and flavouring agents can be as known in the art. However, the sweetner is preferably aspartame and flavouring agent is pineapple flavour.

The present drug delivery system has been found to be suitable for about 2 mg to about 200 mg of Lamotrigine. The swelling agent, particularly the powdered cellulose is taken in an amount varying from about 10% to about 55%, preferably about 30% to about 50% by weight of total formulation. The disintegrating agent, particularly crosslinked polyvinyl pyrrolidone is taken in an amount varying from about 3% to about 6%, preferably about 4% to about 5% by weight of total formulation.

The filler, particularly the α-form of mannitol is taken in an amount varying from about 10% to about 38%, preferably from about 15% to about 32% and more preferably from about 18% to about 30% by weight of the total formulation.

The binder, particularly maltodextrin is taken in an amount varying from about 2% to about 3% by weight of the total formulation.

In accordance to the present invention the Lamotrigine is first adsorbed in an adsorbing medium consisting of the powdered cellulose and the α-form of spray dried mannitol, wherein the cellulose helps in adsorption of the Lamotrigine in the open orifice of the powdered cellulose and mannitol helps in release of adsorbed Lamotrigine.

In accordance to the present invention Lamotrigine and the adsorbing medium are taken in a ratio of about 1:1.66, about 1:23, about 1:1.75 and about 1:1.66 respectively for 200 mg, 2.00 mg, 25 mg and 100 mg of Lamotrigine.

In accordance to further preferred embodiments of the present invention the Lamotrigine is taken in a ratio of about 1:1.05, about 1:14, about 1:1.13, about 1:1.05 with respect to powdered cellulose; the Lamotrigine is taken in a ratio of about 1:0.6, about 1:9.1, about 1:0.6, about 1:0.6 with respect to mannitol; the Lamotrigine is taken in a ratio of about 1:0.16, about 1:1.5, about 1:0.16, about 1:0.16 with respect to crosslinked polyvinyl pyrrolidone; the Lamotrigine is taken in a ratio of about 1:0.1, about 1:0.9, about 1:0.1, about 1:0.1 with respect to maltrodextrin respectively for the formulation containing 200 mg, 2 mg, 25 mg and 100 mg of Lamotrigine.

In accordance to the present invention the relative ratios of Lamotrigine, powdered cellulose, mannitol and crosslinked polyvinyl pyrrolidone are respectively about 1:1.05:0.62:0.16, about 1:14:9.1:1.5, about 1:1.13:0.62:0.16, about 1:1.05:0.62:0.16; and the relative ratios of Lamotrigine, powdered cellulose, mannitol, crosslinked polyvinyl pyrrolidone and maltodextrin are respectively about 1:1.05:0.62:0.16:0.1, about 1:14:9.1:1.5:0.9, about 1:1.13:0.62:0.16:0.1, about 1:1.05:0.62:0.16:0.1 for the formulation containing 200 mg, 2 mg, 25 mg and 100 mg of Lamotrigine.

The present invention also provides a process for manufacturing a mouth dissolvable and meltable, and water dispersable delivery formulation for oral administration comprising the step of adsorbing the antiepileptic drug in the open orifice of the swelling agent and subsequently mixing with the filler followed by granulation with aqueous solution of the binder and mixing with the disintegrating agent and other optional excipients selected from a group comprising lubricants, sweetners and flavouring agents. The swelling agent, the filler, the binder, the disintegrating agent, the lubricating agent, the sweetness and the flavouring agents are as defined and described hereinabove.

As described hereinabove in the brief description of the present invention, this drug delivery system may be used for dual purpose, that is it can be used as mouth dissolving as well as dispersible tablet. The present invention provides a drug delivery system having a designing of mouth dissolving drug delivery capability as well as dispersibility in respective aqueous medium. Thus, the absorption of the drug is enhanced in the present invention. The delivery system dissolves by disintegrating quickly in the patient's mouth without chewing i.e. without the aid of water within a very short span of time. The main advantage of this invention is that it can be taken by patients directly without water, which is also capable of dispersing. Thus it provides accurate dose delivery, avoiding variation of the drug supply from the formulation.

The selected filler mannitol, particularly the α-form of spray dried mannitol not only acts as filling agent, but also acts as melting point depression agent for the Lamotrigine and releasing agent for the Lamotrigine when the formulation is placed in mouth or in water.

The swelling agent, particularly powdered cellulose has been found to enhance the swelling characteristics of the system when it comes in contact with respective aqueous medium.

As stated hereinabove, the pleasant sweet taste can be achieved by using any flavouring agent, but preferably it is achieved by using pineapple flavour.

The drug, particularly Lamotrigine can have any particle size varying from about ≦100 micron to about ≦40 micron for an amount of about 2 mg to about 200 mg. However, the present inventors have found that when Lamotrigine of a definite particle size, preferably about ≦40 micron is taken then it shows better bioabsorption characteristics. The Lamotrigine of particle size of about ≦40 micron has also been found to enhance disintegration and dispersion of the formulation in water, which is easily administrable to the patient. Accordingly, in accordance with preferred embodiment of the present invention the particle size of the Lamotrigine is selected to be about ≦40 micron.

It has been found that the above-said particle size of the Lamotrigine helps in its adsorption within the selected swelling agent powdered cellulose of fibrous texture and plant origin. The adsorption characteristics were also carried out by using hydroxypropyl cellulose, sodium carboxymethyl cellulose and dextrins, but the results with the powdered cellulose were found to be encouraging and hence in accordance to the present invention it becomes the preferred choice for the present drug delivery system.

The particle size of 200 micron of powdered cellulose has been found to give a satisfactory dispersion. On the other hand the same when subjected to dissolution profile in the discriminatory dissolution medium of pH 4.5 showed 84% drug release as against 100% with the formulation wherein drug was embodied in powdered cellulose of average particle size of about 60 micron. This distinct feature of using the natural cellulose with the particle size of about 60 micron gives an excellent In-vitro dissolution profile, which in combination with maltodextrin and a form of spray dried mannitol having specific properties as described hereinabove has been found to result into the desired mouth dissolving drug delivery system.

The low moldability type water soluble low density carbohydrates, as preferred for the present invention are mannitol, glucose, sucrose and xylitol. The low moldability type low density carbohydrate 1, 2, 3, 4, 5, 6-hexanehexol is spray dried of average particle size of about 150 micron in diameter having dissolution time in water with a ratio of 1:30 by weight with respect to water, flow time of 8 seconds and Haussner ratio of 1.16.

The particularly α-form of mannitol (Pearlitol®) having the above particle size, out of α, β and δ forms, showed novel melting point depression ability. The further studies showed there is a distinct depression of melting point of Lamotrigine when used in combination with this a form of mannitol.

The depression of melting point of Lamotrigine was achieved from 215° C. to 190° C. on employing mannitol. This feature of melting point depression of Lamotrigine has resulted in designing the present mouth dissolving and dispersible Lamotrigine tablets having concentration of 2 to 200 mg of the drug per unit dose. When the dissolution time of the α-form of spray dried mannitol is compared with the standard and granulated mannitol, it gave following gradation:—

Spray dried α-mannitol<standard mannitol<granulated mannitol

which quantified as 5 sec, 35 sec and 100 seconds respectively.

The combination of melting point depression characteristic and negative heat of solution characteristic of the selected a form of spray dried mannitol has also been found to result in cooling effect to the present drug delivery system.

As stated hereinabove, while designing the mouth dissolving and dispersible drug delivery system, the disintegrating agents are selected from the group consisting of crosslinked polyvinyl pyrrolidone, corn starch, Acacia, crosscarmellose sodium, low viscosity hydroxypropyl cellulose, xanthan gum. Preferably the disintegrating agent for the present invention are crosslinked polyvinyl pyrrolidone and crosscarmellose sodium. The crosslinked polyvinyl pyrrolidone (Polyplasdone XL®) may be used as disintegrating agent either alone or in combination with powdered cellulose which is also used as swelling agent in the present invention. The crosslinked polyvinyl pyrrolidone due to its higher molecular weight and crosslinked structure is used as low as possible 2 to 10%, more preferably between 4 to 8%, most preferably between 4 to 5% by weight of the total formulation. The presence of crosslinked polyvinyl pyrrolidone in combination with other selected ingredients also helps in achieving a particular swelling, that is four times swelling of Lamotrigine. The combined effect of powdered cellulose, maltodextrin, α-form of mannitol along with crosslinked polyvinyl pyrrolidone in the above defined ratios has been found to give a tremendous volume expansion during processing which in contact with aqueous medium swells to the range of about 4 times. This achieved feature was helped in arriving at dual drug delivery aspects of the present formulation, which can be in used as mouth dissolving as well as dispersible tablets. The powdered cellulose of about 60 micron particle size and α-form of the spray dried mannitol first adsorbs Lamotrigine on the fibrous orifice and releases easily after drying, in contact with aqueous medium. This observation was evaluated by checking In-vitro dissolution profile of the dosage form in two discriminatory dissolution media of pH 4.5 and pH 7.2. The pH 7.2 was selected with the aim of getting pH of the saliva. The In-vitro dissolution at the end of 30 minutes following USP dissolution apparatus was about 100%. This was similar to that in the pH of 4.5. The In-vitro dissolution in pH 7.2 and pH 4.5 confirms that this design of drug delivery system can be used as both—mouth dissolving and dispersible system with estimated satisfactory bioavailability.

In accordance to the present invention, a novel dual purpose mouth dissolving and dispersible tablet formulation has been designed with an improved In-vitro dissolution profile in discriminatory dissolution medium of pH 4.5 and 7.2. With this system an accurate drug delivery to the patient is assured by avoiding dispersion in water and then administration of it. Herein the anti-epileptic drug was processed following dry and wet granulation method. The wet granulation was found to be superior to dry granulation in the present invention with the cellulose, having described quality of average particle size of about 60 micron. The anti-epileptic drug, particularly Lamotrigine in an amount varying from 2 to 200 mg of a particular particle size of about ≦40 micron embodied in powdered cellulose of said particle size, which first adsorbs the drug in its open orifice which is further covered with filler.

While designing the manufacturing process, both the options of wet granulation and direct compression were evaluated. Based on higher In-vitro dissolution profile following wet granulation technology, using the powdered cellulose of average particle size of about 60 micron as described earlier was preferred. The wet granules obtained by this process were dried and thereafter lubricated with the lubricating agent followed by optional addition of sweetner and flavouring agents and compressed to tablets. The resulted tablets were found to have dual properties of mouth dissolving and meltability, and water dispersibility.

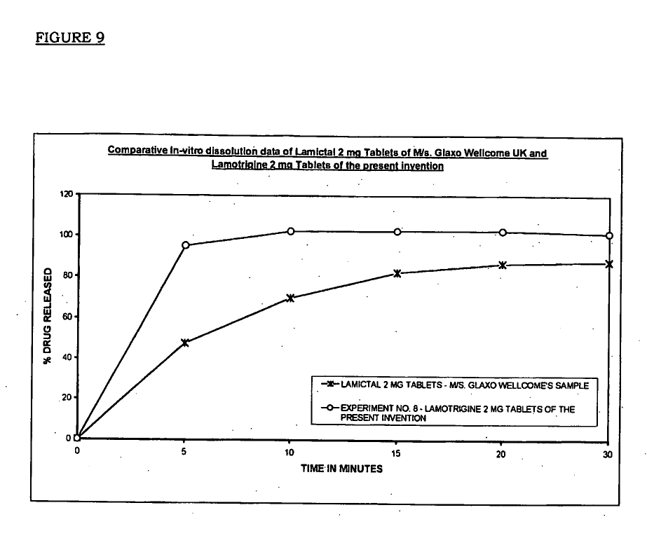

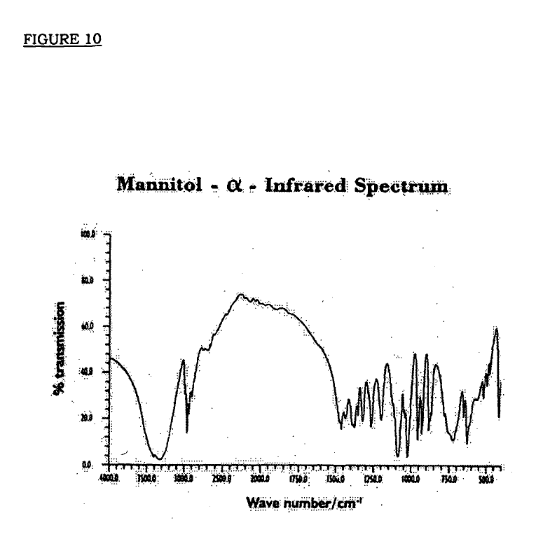

The above tablets was checked for In-vitro dissolution in the discriminatory dissolution medium of pH 4.5, comparing that of Lamictal dispersible tablets of M/s. Glaxo Wellcome, UK. The In-vitro dissolution profile as referred in FIGS. 8 and 9 shows improvement in the drug release pattern of the present invention.

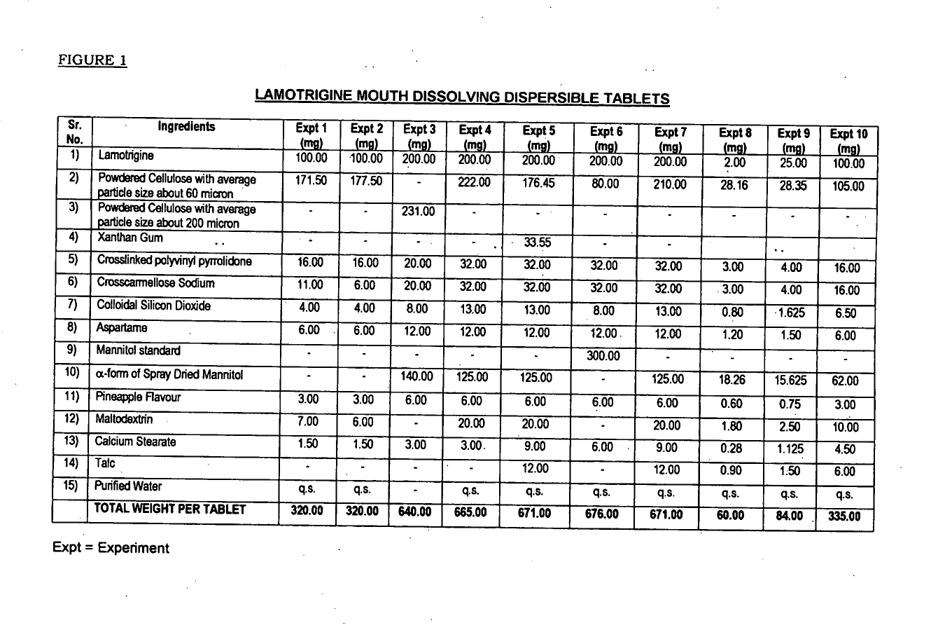

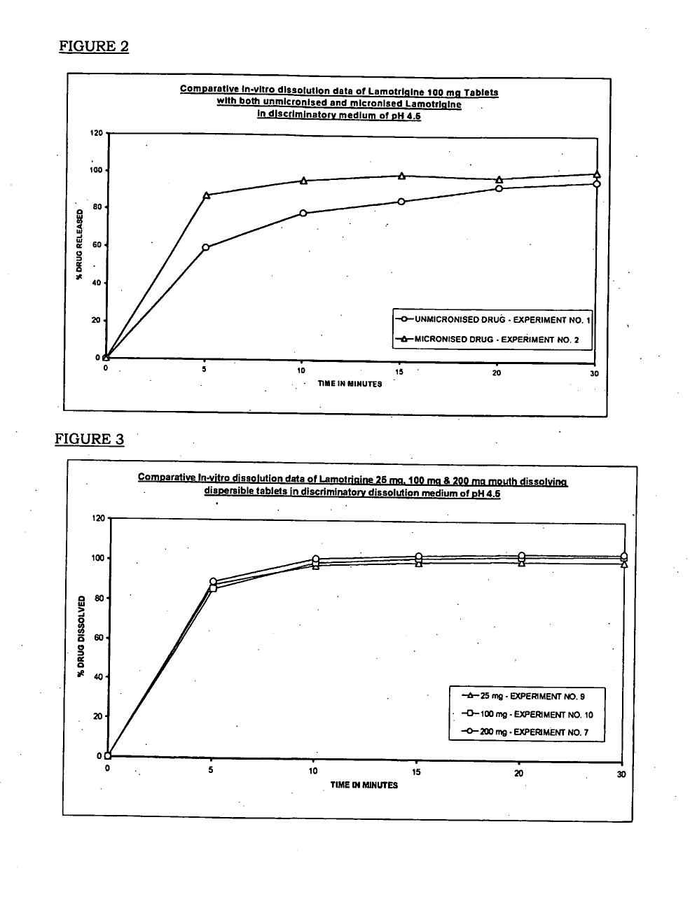

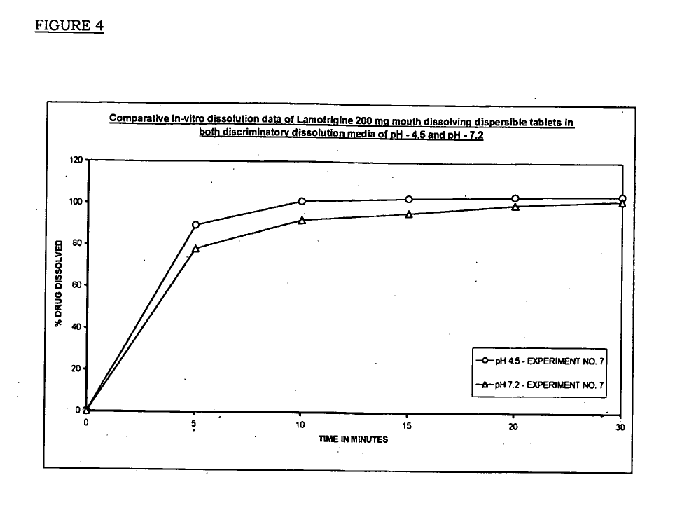

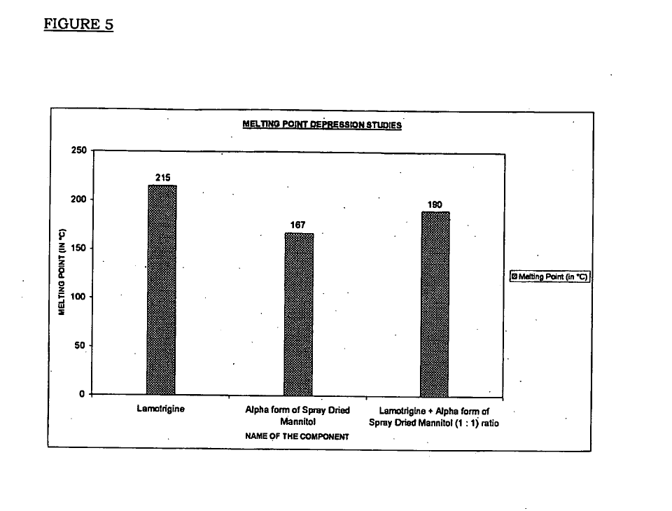

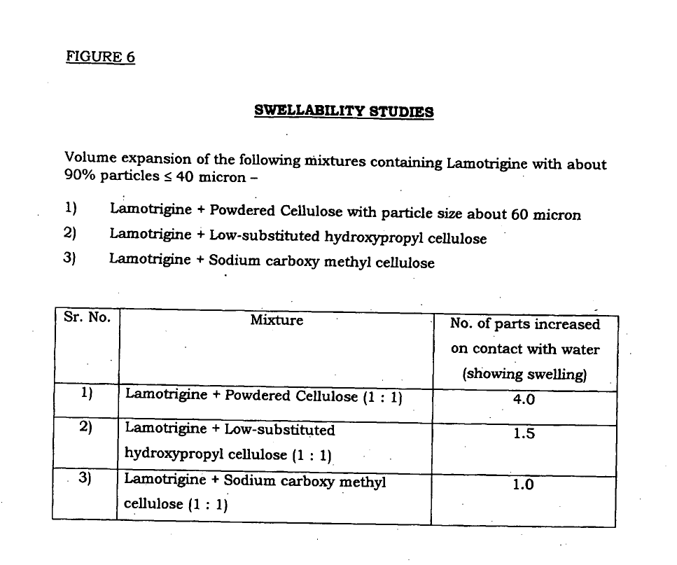

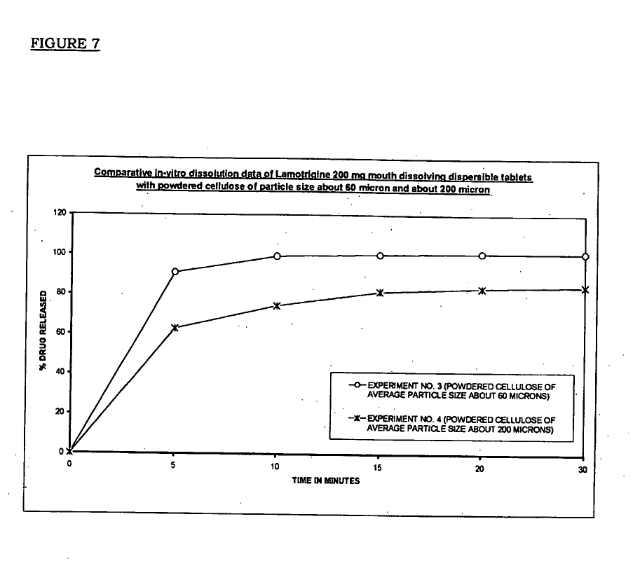

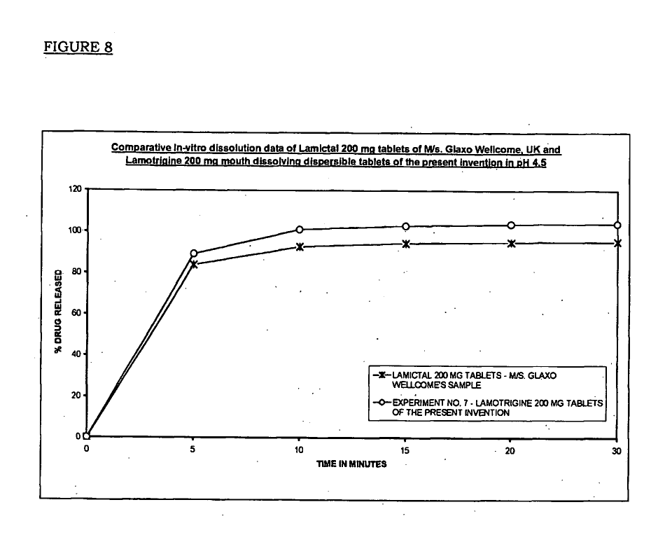

It has been surprisingly found that the drug delivery formulations prepared in accordance with the present invention comprising the defined selected ingredients in their selected weight ratios as defined hereinabove and elaborated hereinbelow in the experimental studies [FIG. 1] show better In-vitro dissolution profile with micronised particles of the Lamotrigine having particle size of about ≦40 microns [FIG. 2], show similar In-vitro dissolution profile of the present drug delivery system in 25 mg, 100 mg and 200 mg tablet forms indicating that it remains uniform and reproducible in various tablet forms and is not altered with variation of Lamotrigine's strength [FIG. 3], with variation in pH [FIG. 4], melting point of Lamotrigine reduces significantly [FIG. 5], show significant increase in swellability or volume expansion due to use of powdered cellulose as swelling agent [FIG. 6], better In-vitro dissolution profile due to use of powdered cellulose with an average particle size of about 60 microns [FIG. 7], better In-vitro dissolution profile when compared with selected standard drug delivery system known in the art [FIG. 8 and FIG. 9]. FIG. 10 confirms that the mannitol used is α-form of mannitol.

### EXPERIMENTAL STUDIES

The presently disclosed formulations were prepared in accordance to the present method of preparation as described hereinabove and having relative concentrations of various ingredients as described hereinabove and particularly as shown in FIG. 1. The In-vitro dissolution profile of the powdered cellulose of plant origin with an average particle size of about 60 micron and about 200 micron was determined in discriminatory dissolution media of pH 4.5, which respectively has shown 100% and 84% dissolution at the end of 30 minutes as shown in FIG. 7. It was also observed that the micronised Lamotrigine drug having particle size of about ≦40 microns gives significantly higher dissolution profile than that with unmicronised Lamotrigine as seen in FIG. 2.

The another aspect of the present invention is the ratio of crosslinked polyvinyl pyrrolidone and α-form of spray dried mannitol with respect to the drug as described hereinabove. This is of significance, as there exists a competition between mannitol and crosslinked polyvinyl pyrrolidone for the aqueous fluid that penetrates into the tablet when the present dosage form comes in contact with the disintegrating fluid namely saliva or water. Accordingly, the ratio of crosslinked polyvinyl pyrrolidone to the α-form of spray dried mannitol is critical to have good disintegration without affecting the mouth dissolving property of the present dosage form. In the present invention the ratio of crosslinked polyvinyl pyrrolidone with respect to α-form of spray dried mannitol is most preferably about 1:4. The concentration of crosslinked polyvinyl pyrrolidone has been found to be effective at 3 to 5% by weight of the total formulation, as there was no significant improvement in disintegration time by increasing the con centration of the same. It has been observed that all the formulations have satisfactory dissolution profile but do not possess the mouth dissolving properties. Such formulations are of experiments 1, 2 and 6, wherein particularly selected filler a form of mannitol is absent establishing that it also enhances mouth dissolving property of the present drug delivery system in-addition to providing melting point depression of Lamotrigine which further helps in mouth dissolving property of the present drug delivery system.

The formulations were prepared having compositions as mentioned in FIG. 1. For experimental purpose only different combinations of said excipients were used. However, the combinations of experiments 7, 8, 9 and 10 of compositions as shown in FIG. 1, were found to have satisfactory mouth dissolving and dispersion properties. The In-vitro dissolution profile of these experiments as shown in FIG. 3 and FIG. 9 were found about 100% in the discriminatory dissolution medium of pH 4.5 using USP dissolution apparatus. The experiment No. 7 having highest Lamotrigine content of 200 mg was also subjected to In-vitro dissolution test at pH 4.5 and pH 7.2 in the USP apparatus and the drug release at the end of 30 minutes was found to be 100% as depicted in the FIG. 4.

The swellability characteristics of different excipients used in the present invention were evaluated to select the best. The different excipients selected for evaluating the swellability include fibrous powdered cellulose of natural origin with average particle size of about 60 microns, low substituted hydroxypropyl cellulose and sodium carboxymethyl cellulose. The swellability characteristic was evaluated by mixing each of the above mentioned excipient individually with that of the micronised Lamotrigine with particle size of about ≦40 microns in 1:1 ratio and wetted sufficiently with equal quantity of water which resulted in swelling of the mix as shown in FIG. 6 with the following order: Fibrous powdered cellulose of natural origin with average particle size of about 60 micron>low substituted hydroxypropyl cellulose>sodium carboxymethyl cellulose, i.e. 4>1.5>1 respectively, indicating the number of parts swelled on contact with water (FIG. 6).

The presently disclosed formulations prepared in accordance to the present method of preparation as described hereinabove and having said relative concentrations of various ingredients have shown better dispersion in water with a disintegration time of about ≦35 seconds. The formulations exhibited good mouth dissolving properties too. But as stated hereinabove, the formulation with powdered cellulose of particle size of about 200 micron showed a very poor dissolution i.e. 84% at the end of 30 minutes, whereas the formulation with powdered cellulose of average particle size of about 60 micron showed 100% at the end of 30 minutes. Hence, substantiating the selection of latter formulation as the most preferable one than the former. The In-vitro dissolution profile obtained for the preferred embodiment containing 2 mg and 200 mg of Lamotrigine of the present invention was compared with the profile of the Lamictal of the same strength i.e. 2 mg and 200 mg in discriminatory dissolution medium of pH 4.5. While the clinical studies are being planned, the In-vitro dissolution studies of the present invention so far exhibited improved and satisfactory release patterns at the end of 30 minutes as shown in FIGS. 8 and 9.

All the above experiments were carried out in the laboratory at a temperature less than 25° C. with relative humidity less than 60%.

It will be obvious to the persons skilled in the art after referring to the forgoing description and the forgoing examples and their findings that the drug delivery systems of the forgoing examples, particularly of the experiments 7, 8, 9 and 10 are the best embodiments of the present invention. Further, the present invention has been described with the help of accompanying figures and the forgoing examples, which are not intended to limit scope of the present invention. It is obvious to the persons skilled in the art that variations are possible without deviating from the scope of the present invention.

1. A mouth dissolvable and meltable, and water dispersable delivery formulation for oral administration consisting of an antiepileptic drug, one or more swelling agents, one or more of fillers, one or more of disintegrating agents, and one or more of binders, characterized in that
said an antiepileptic drug is preferably Lamotrigine for the antiepileptic treatment;
said swelling agent is selected from the group comprising powdered cellulose and crosscarmellose sodium;
said filler is selected from the group comprising spray dried mannitol, glucose, sorbitol, maltose and fructose;
said disintegrating agent is selected from the group comprising crosslinked polyvinyl pyrrolidone, corn starch, acacia, crosscarmellose sodium and xanthan gum; and
said binder is maltodextrin; and
wherein said formulation may optionally comprises one or more of other excipients selected from the group comprising lubricants, sweeteners and flavouring agent.

2. A formulation as claimed in claim 1, characterized in that said Lamotrigine is micronised Lamotrigine.

3. A formulation as claimed in claim 2, characterized in that said Lamotrigine has particle size of about ≦100 micron, preferably about ≦70 micron, most preferably about ≦40 micron.

4. A formulation as claimed in claim 1, characterized in that said swelling agent is powdered cellulose which is preferably fibrous and derived from plant.

5. A formulation as claimed in claim 1, characterized in that said powdered cellulose has average particle size of about 200 microns, most preferably about 60 microns.

6. A formulation as claimed in claim 5, characterized in that said powdered cellulose has bulk density of about 0.1 to 0.28 gm/ml, pH of about 5 to 7.5, ether and water soluble substance of about ≦0.15% and about ≦1.5% respectively, with loss on drying about ≦6%, and degree of polymerisation of about 440 to 2250.

7. A formulation as claimed in claim 1, characterized in that said filler is mannitol, preferably α form of spray dried mannitol.

8. A formulation as claimed in claim 7, characterized in that said α-form of mannitol has mean particle size diameter of about 150 micron and Haussner number of about 1.16, having dissolution time in water with a ratio of 1:30 with respect to water in w/w, not more than 5 seconds at 20° C.

9. A formulation as claimed in claim 1, characterized in that said disintegrating agent consists of crosslinked polyvinyl pyrrolidone or crosscarmellose sodium or both.

10. A formulation as claimed in claim 1, characterized in that said maltodextrin has dextrose equivalent of about 13 and protein content maximum of about 0.15%, carbohydrate composition of glucose of about 1%, maltose of about 2%, and oligo and polysaccharides of about 97%.

11. A formulation as claimed in claim 1, characterized in that said lubricating agent is selected from a group comprising calcium stearate, colloidal silicon dioxide and talc.

12. A formulation as claimed in claim 1, characterized in that said Lamotrigine is taken in an amount varying from about 2 mg to about 200 mg.

13. A formulation as claimed in claim 1, characterized in that said powdered cellulose is taken in an amount varying from about 10% to about 55%, preferably about 30% to about 50% by weight of total formulation.

14. A formulation as claimed in claim 1, characterized in that said crosslinked polyvinyl pyrrolidone is taken in an amount varying from about 3% to about 6%, preferably about 4% to about 5% by weight of total formulation.

15. A formulation as claimed in claim 1, characterized in that said α-form of mannitol is taken in an amount varying from about 10% to about 38%, preferably from about 15% to about 32% and more preferably from about 18% to about 30% by weight of the total formulation.

16. A formulation as claimed in claim 1, characterized in that said maltodextrin is taken in an amount varying from about 2% to about 3% by weight of the total formulation.

17. A formulation as claimed in claim 1, characterized in that said Lamotrigine and adsorbing medium are taken in a ratio of about 1:1.66, about 1:23, about 1:1.75 and about 1:1.66 respectively for 200 mg, 2.00 mg, 25 mg and 100 mg of said Lamotrigine.

18. A formulation as claimed in claim 17, characterized in that said adsorbing medium consists of said powdered cellulose and said α-form of spray dried mannitol, wherein said cellulose helps in adsorption and mannitol helps in release of Lamotrigine.

19. A formulation as claimed in claim 1, characterized in that said Lamotrigine is taken in a ratio of about 1:1.05, about 1:14, about 1:1.13, about 1:1.05 with respect to powdered cellulose respectively for the formulation containing 200 mg, 2 mg, 25 mg and 100 mg of Lamotrigine.

20. A formulation as claimed in claim 1, characterized in that said Lamotrigine is taken in a ratio of about 1:0.6, about 1:9.1, about 1:0.6, about 1:0.6 with respect to mannitol respectively for the formulation containing 200 mg, 2 mg, 25 mg and 100 mg of Lamotrigine.

21. A formulation as claimed in claim 1, characterized in that said Lamotrigine is taken in a ratio of about 1:0.16, about 1:1.5, about 1:0.16, about 1:0.16 with respect to crosslinked polyvinyl pyrrolidone respectively for the formulation containing 200 mg, 2 mg, 25 mg and 100 mg of Lamotrigine.

22. A formulation as claimed in claim 1, characterized in that said Lamotrigine is taken in a ratio of about 1:0.1, about 1:0.9, about 1:0.1, about 1:0.1 with respect to maltrodextrin respectively for the formulation containing 200 mg, 2 mg, 25 mg and 100 mg of Lamotrigine.

23. A formulation as claimed in claim 1, characterized in that said Lamotrigine, powdered cellulose, mannitol and crosslinked polyvinyl pyrrolidone are respectively taken in a ratio of about 1:1.05:0.62:0.16, about 1:14:9.1:1.5, about 1:1.13:0.62:0.16, about 1:1.05:0.62:0.16 for the formulation containing 200 mg, 2 mg, 25 mg and 100 mg of Lamotrigine.

24. A formulation as claimed in claim 1, characterized in that said Lamotrigine, powdered cellulose, mannitol, crosslinked polyvinyl pyrrolidone and maltodextrin are respectively taken in a ratio of about 1:1.05:0.62:0.16:0.1, about 1:14:9.1:1.5:0.9, about 1:1.13:0.62:0.16:0.1, about 1:1.05:0.62:0.16:0.1 for the formulation containing 200 mg, 2 mg, 25 mg and 100 mg of Lamotrigine.

25. A process for manufacturing a mouth dissolvable and meltable, and water dispersable delivery formulation for oral administration comprising the step of adsorbing said antiepileptic drug in the open orifice of said swelling agent and subsequently mixing with said filler followed by granulation with aqueous solution of said binder and mixing with said disintegrating agent and other optional excipients selected from the group consisting of lubricants, sweeteners and flavouring agents.
